(19) United States
(12) Patent Application Publication
(10) Pub. No.: US 2024/0412834 A1
(43) Pub. Date: Dec. 12, 2024

(54) Methods and Systems for Heart Monitoring

(71) Applicant: READMYRHYTHM INC., Searcy, AR (US)

(72) Inventor: Alissa P. Manfredi, Searcy, AR (US)

(21) Appl. No.: 18/738,579

(22) Filed: Jun. 10, 2024

Related U.S. Application Data
(60) Provisional application No. 63/507,258, filed on Jun. 9, 2023.

(51) Int. Cl. G16H 15/00; A61B 5/00; A61B 5/308; A61B 5/318; G16H 40/63

(52) U.S. Cl. CPC G16H 15/00; A61B 5/0006; A61B 5/308; A61B 5/318; G16H 40/63

(57) ABSTRACT

A method for heart monitoring includes receiving data corresponding to an electrocardiogram taken by a wearable device and transmitting data corresponding to an interpretation of the electrocardiogram by an interpretation provider. A referral to a medical service provider may be sent in response to the interpretation of the electrocardiogram indicating an irregularity.

### CROSS-REFERENCE TO RELATED APPLICATION

The present application is related and has right of priority to U.S. Provisional Application No. 63/507,258, which was filed in the U.S. Patent & Trademark Office on Jun. 9, 2023 and is incorporated by reference herein for all purposes.

### FIELD OF THE INVENTION

The present subject matter relates generally to systems and methods for heart monitoring, such as with electrocardiograms.

### BACKGROUND OF THE INVENTION

Electrocardiograms (ECGs) are frequently used by medical service providers to monitor heart health. The pattern of electrical activity shown in the electrocardiogram can assist with detecting cardiac abnormalities, including cardiac rhythm disturbances, inadequate coronary artery blood flow, and electrolyte disturbances. Conventionally, ECGs are taken with twelve leads attached to a prone patient; however, such systems are bulky and frequently require medical personnel to oversee recording signals. Recently, wearable devices, such as smartwatches, capable of more comfortably and easily taking ECGs have become available; however, reviewing ECGs taken from wearable devices, such as smartwatches, can be difficult for medical service providers.

Accordingly, improved systems and methods for heart monitoring would be useful.

### BRIEF DESCRIPTION OF THE INVENTION

Aspects and advantages of the invention will be set forth in part in the following description, or may be apparent from the description, or may be learned through practice of the invention.

In an example embodiment, a method for interpreting electrocardiograms includes: receiving data corresponding to an electrocardiogram taken by a wearable device; transmitting data corresponding to an interpretation of the electrocardiogram by an interpretation provider; and transmitting data corresponding to a referral to a medical service provider in response to the interpretation of the electrocardiogram indicating an irregularity.

In another example embodiment, a computing system includes one or more processors and one or more non-transitory computer-readable media that store instructions that, when executed by the one or more processors, cause the computing system to perform operations. The operations include: receiving data corresponding to an electrocardiogram taken by a wearable device; transmitting data corresponding to an interpretation of the electrocardiogram by an interpretation provider; and transmitting data corresponding to a referral to a medical service provider in response to the interpretation of the electrocardiogram indicating an irregularity.

In another example embodiment, a method for interpreting electrocardiograms includes: associating a wearable device with a medical service provider; receiving data corresponding to a plurality of electrocardiograms over a period of time, each of the plurality of electrocardiograms taken by the wearable device, the period of time being no less than sixteen days; receiving data corresponding to an interpretation of each of the plurality of electrocardiograms by an interpretation provider during the period of time; and transmitting data corresponding to a billable report for the interpretation of each of the plurality of electrocardiograms to the medical service provider after the period of time.

In another example embodiment, a computing system includes one or more processors and one or more non-transitory computer-readable media that store instructions that, when executed by the one or more processors, cause the computing system to perform operations. The operations include: associating a wearable device with a medical service provider; receiving data corresponding to a plurality of electrocardiograms over a period of time, each of the plurality of electrocardiograms taken by the wearable device, the period of time being no less than sixteen days; receiving data corresponding to an interpretation of each of the plurality of electrocardiograms by an interpretation provider during the period of time; and transmitting data corresponding to a billable report for the interpretation of each of the plurality of electrocardiograms to the medical service provider after the period of time.

These and other features, aspects and advantages of the present invention will become better understood with reference to the following description and appended claims. The accompanying drawings, which are incorporated in and constitute a part of this specification, illustrate embodiments of the invention and, together with the description, serve to explain the principles of the invention.

### BRIEF DESCRIPTION OF THE DRAWINGS

A full and enabling disclosure of the present invention, including the best mode thereof, directed to one of ordinary skill in the art, is set forth in the specification, which makes reference to the appended figures.

FIG. 1 is a schematic view of a system for heart monitoring according to an example embodiment of the present subject matter.

FIG. 2 is a schematic view of certain components of the example system of FIG. 1.

FIG. 3 is a flow diagram of a method for heart monitoring according to an example embodiment of the present subject matter.

FIG. 4 is a flow diagram of another method for heart monitoring according to an example embodiment of the present subject matter.

### DETAILED DESCRIPTION

Reference now will be made in detail to embodiments of the invention, one or more examples of which are illustrated in the drawings. Each example is provided by way of explanation of the invention, not limitation of the invention. In fact, it will be apparent to those skilled in the art that various modifications and variations can be made in the present invention without departing from the scope or spirit of the invention. For instance, features illustrated or described as part of one embodiment can be used with another embodiment to yield a still further embodiment. Thus, it is intended that the present invention covers such modifications and variations as come within the scope of the appended claims and their equivalents.

As used herein, the terms “includes” and “including” are intended to be inclusive in a manner similar to the term “comprising.” Similarly, the term “or” is generally intended to be inclusive (i.e., “A or B” is intended to mean “A or B or both”). Approximating language, as used herein throughout the specification and claims, is applied to modify any quantitative representation that could permissibly vary without resulting in a change in the basic function to which it is related. Accordingly, a value modified by a term or terms, such as “about,” “approximately,” and “substantially,” are not to be limited to the precise value specified. In at least some instances, the approximating language may correspond to the precision of an instrument for measuring the value. For example, the approximating language may refer to being within a ten percent (10%) margin.

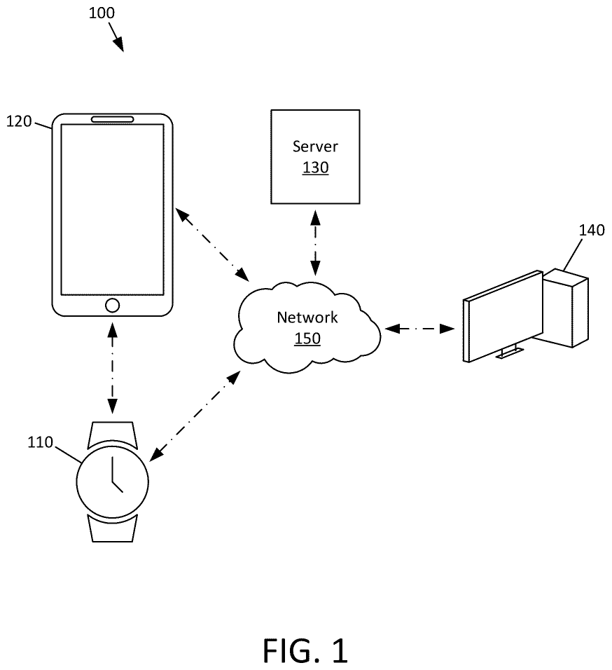

FIG. 1 is schematic view of a system 100 for heart monitoring. As shown in FIG. 1, system 100 may include a wearable device 110. Wearable device 110 may be a smartwatch, such as an Apple Watch, a Samsung Galaxy Watch, a Withings ScanWatch, or a Fitbit, or other type of wearable device. Thus, a patient may don wearable device 110 such that wearable device 110 is positioned on a wrist or other body part of the patient. As discussed in greater detail below, wearable device 110 may include an electrical heart sensor configured for taking an electrocardiogram (ECG) when the wearable device 110 is worn by the patient.

System 100 may also include one or more of a personal computing device 120, a server 130, and an interpretation provider computing device 140. Wearable device 110 may communication with one or more of personal computing device 120, server 130, and interpretation provider computing device 140 via a network 150. Thus, e.g., wearable device 110 may transmit data to and/or receive data from computing device 120, server 130, and/or interpretation provider computing device 140 via network 150.

As an example of certain aspects of the operation of system 100, a patient may use wearable device 110 to take an ECG. Wearable device 110 may then transmit the ECG for analysis. Moreover, the ECG from wearable device 110 may be transmitted to interpretation provider computing device 140 via network 150. For instance, wearable device 110 may be configured for communication via a cellular network, such as via LTE and/or UMTS bands, and wearable device 110 may transmit the ECG to interpretation provider computing device 140 via the cellular network connection. In certain example embodiments, the ECG from wearable device 110 may first be transmitted to personal computing device 120. Personal computing device 120 may be a smartphone, tablet, etc. of the user of wearable device 110 in communication with wearable device 110 via a short-range wireless connection, such as Bluetooth® connection, between personal computing device 120 and wearable device 110. Personal computing device 120 may then transmit the ECG from wearable device 110 to interpretation provider computing device 140 via network 150, such as via a cellular network connection and/or a Wi-Fi connection. Server 130 may also assist with connecting wearable device 110 and interpretation provider computing device 140 to facilitate transmission of the ECG from wearable device 110 to interpretation provider computing device 140. At interpretation provider computing device 140, a cardiologist, an electrophysiologist, a cardiac nurse practitioner, a cardiac physician assistant, etc. can view and interpret the ECG from wearable device 110. The interpretation of the ECG may then be transmitted to the patient, e.g., to wearable device 110 and/or computing device 120, via network 150. The patient may then advantageously utilize the interpretation of the ECG to make an informed medical decision regarding cardiac care.

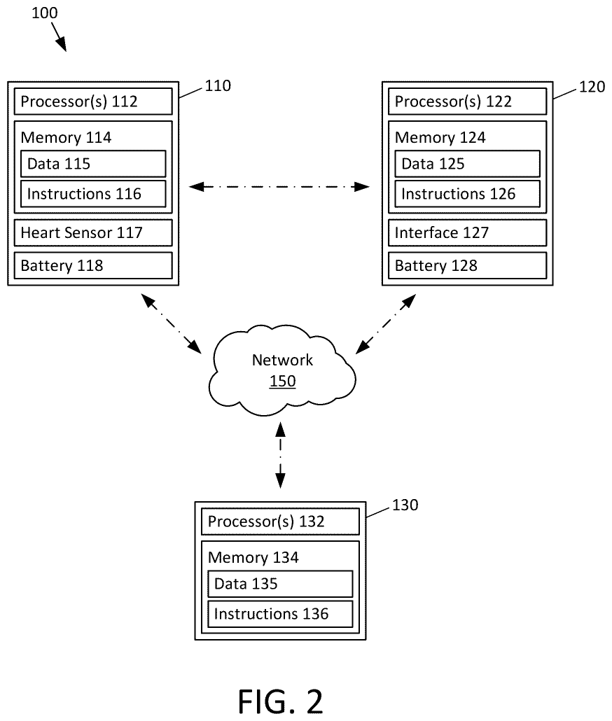

Turning to FIG. 2, wearable device 110 may include one or more processors 112 and one or more memory devices 114 (hereinafter referred to as “memories 114”). The one or more memories 114 store information accessible by the one or more processors 112, including instructions 116 that may be executed and data 115 usable by the one or more processors 112. The one or more memories 114 may be of any type capable of storing information accessible by the one or more processors 112, including a computing device-readable medium. The memory is a non-transitory medium, such as a hard-drive, memory card, optical disk, solid-state, tape memory, or the like. The one or more memories 114 may include different combinations of the foregoing, whereby different portions of the instructions and data are stored on different types of media. The one or more processor 112 may be any conventional processors, such as commercially available CPUs. Alternatively, the one or more processors 112 may be a dedicated device, such as an ASIC or other hardware-based processor.

Instructions 116 may be any set of instructions to be executed directly (such as machine code) or indirectly (such as scripts) by the one or more processors 112. For example, the instructions 116 may be stored as computing device code on the computing device-readable medium of the one or more memories 114. In that regard, the terms “instructions” and “programs” may be used interchangeably herein. Instructions 116 may be stored in object code format for direct processing by the processor or in any other computing device language, including scripts or collections of independent source code modules that are interpreted on demand or compiled in advance. Wearable device 110 may include various programs and/or applications within instructions 116. For example, instructions 116 may include a clock application, a calendar application, an email application, a text messaging application, etc.

Data 115 may be retrieved, stored, or modified by the one or more processors 112 in accordance with the instructions 116. For instance, data 115 of the one or more memories 114 may store information from an electrical heart sensor 117, such as a single lead electrical heart sensor, and other elements of wearable device 110. Thus, wearable device 110 may include multiple processors, computing devices, and/or memories disposed within a common physical housing. A battery 118 may provide electrical energy for powering processors 112, memories 114, electrical heart sensor 117, and other components of wearable device 110.

Personal computing device 120 may also include one or more processors 122 and one or more memories 124. The one or more memories 124 store information accessible by the one or more processors 122, including instructions 126 that may be executed and data 125 usable by the one or more processors 122. Reference is made to the above description of component of wearable device 110, which is also applicable to corresponding components of personal computing device 120. Repeated description of the components of personal computing device 120 is omitted for the sake of brevity. Personal computing device 120 may include multiple processors, computing devices, and/or memories disposed within a common physical housing. A battery 128 may provide electrical energy for powering processors 122, memories 124, an interface 127 (such as a touchscreen), and other components of personal computing device 120. Interpretation provider computing device 140 may also include similar components to that described above for personal computing device 120.

Server 130 may also include one or more processors 132 and one or more memories 134. The one or more memories 134 store information accessible by the one or more processors 132, including instructions 136 that may be executed and data 135 usable by the one or more processors 132. However, server 130 may actually include multiple processors, computing devices, and/or memories that may or may not be stored within a common physical housing. Similarly, the one or more memories 134 may be a hard drive or other storage media located in a housing different from that of the processor(s) 132. Accordingly, server 130 will be understood to include a collection of processor(s) and one or more memories that may or may not operate in parallel.

The components of system 100, including wearable device 110, personal computing device 120, server 130, and interpretation provider computing device 140, may include wireless communication systems for connecting to network 150. The wireless communication systems may include an antenna and a chipset configured to communicate according to one or more wireless communication protocols, such as Bluetooth, communication protocols described in IEEE 802.11, GSM, CDMA, UMTS, EV-DO, WiMAX, LTE, Zigbee, dedicated short range communications (DSRC), radio frequency identification (RFID) communications, etc. It should be appreciated that the components of system 100, including wearable device 110, personal computing device 120, server 130, and interpretation provider computing device 140, may also communicate via wired connection on network 150.

As described above, system 100 may be configured for taking ECGs via wearable device 110 and interpreting the ECGs at interpretation provider computing device 140. In addition, system 100 may include features for referring user of wearable device 110 to a medical service provider, such as a cardiologist and/or an electrophysiologist, in response to the interpretation of the ECG indicating an irregularity. For example, the interpretation of the ECG may indicate one or more of: (1) a cardiac rhythm disturbance, such as atrial fibrillation or ventricular tachycardia; (2) inadequate coronary artery blood flow, such as myocardial ischemia or myocardial infarction; and (3) electrolyte disturbances, as hypokalemia and hyperkalemia. System 100 may transmit a referral to the medical service provider when the interpretation of the ECG indicates the irregularity in order to advantageously direct the user of wearable device 110 to the medical service provider for additional testing, medical care, etc. The system 100 may also transmit the ECG and/or the interpretation of the ECG to the medical service provider in order to advantageously deliver the background heat monitoring information regarding the user of wearable device 110 to the medical service provider and thereby assist with follow-up treatment and/or testing.

System 100 may further include features for providing a billable report of ECGs taken with wearable device 110 for the medical service provider. Moreover, system 100 may take a plurality of ECGs via wearable device 110 over a period of time. The period of time may correspond to a billable period of time, such as no less than sixteen (16) days. System 100 may interpret the ECGs at interpretation provider computing device 140 during the period of time, and the medical service provider may receive the interpretation of each of the ECGs during the period of time. System 100 may also generate a billable report for interpreting the ECGs after the period of time. As may be seen from the above, system 100 may provide a billable report for ECGs taken with wearable device 110, e.g., via a single lead electrical heart sensor of wearable device 110. Thus, e.g., the medical service provider may advantageously submit a claim for the billable report to an insurance provider for the user of wearable device 110.

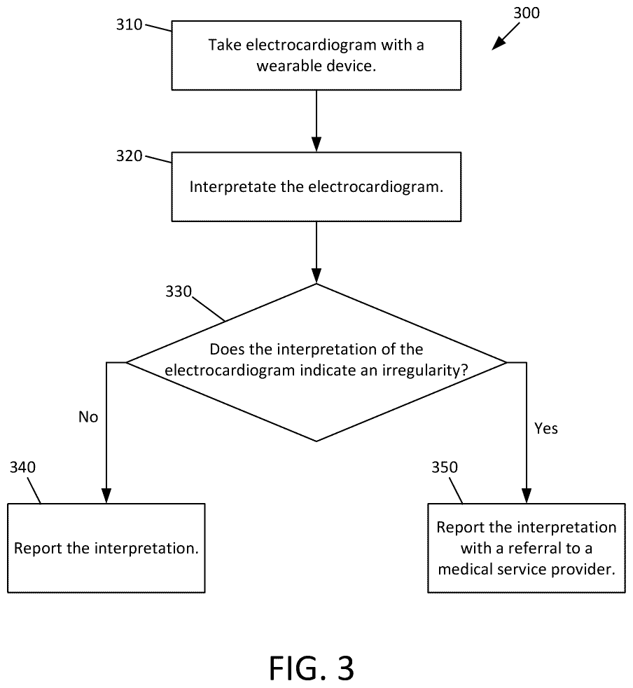

FIG. 3 is a flow diagram of a method 300 for heart monitoring according to an example embodiment of the present subject matter. Method 300 will generally be described with reference to system 100 described with reference to FIGS. 1 and 2. For instance, method 300 may be at least partially executed by system 100, such as by one or more of wearable device 110, personal computing device 120, server 130, and interpretation provider computing device 140. However, method 300 may be suitable for use with any other suitable type of system. In addition, although FIG. 3 depict steps performed in a particular order for purposes of illustration and discussion, the methods and algorithms discussed herein are not limited to any particular order or arrangement. One skilled in the art, using the disclosures provided herein, will appreciate that various steps of the methods and algorithms disclosed herein can be omitted, rearranged, combined, and/or adapted in various ways without deviating from the scope of the present disclosure.

Prior to 310, method 300 may include receiving data corresponding to user information for wearable device 110. For example, the user information for wearable device 110 may include one or more of a location of the user of wearable device 110, an age of the user, a sex of the user, an address of the user, a zip code of the user, a medical history of the user, etc. Such user information may assist with interpretation of electrocardiograms (ECGs) and/or providing medical treatment for the user of wearable device 110.

At 310, an ECG may be taken with wearable device 110. For example, electrical heart sensor 117 may measure electrical activity of the heart of the user of wearable device 110, e.g., via a single lead of electrical heart sensor 117 contacting the skin of the user. The ECG may be taken at 310 in response to a user input at wearable device 110. Thus, the user of wearable device 110 may select when the ECG is taken at 310. In alternative example embodiments, the ECG may be automatically taken at 310, e.g., according to a predetermined schedule, in response to a sensed parameter, etc. Data corresponding to the ECG taken by wearable device 110 at 310 may be transmitted from wearable device 110 to various components of system 100, such as personal computing device 120, server 130, and/or an interpretation provider computing device 140, via network 150. The data corresponding to the ECG taken by wearable device 110 at 310 may be received at interpretation provider computing device 140.

At 320, the ECG taken by wearable device 110 at 310 may be interpreted. For example, a cardiologist, an electrophysiologist, a cardiac nurse practitioner, a cardiac physician assistant, or other interpretation provider may view and interpret the ECG from wearable device 110 at interpretation provider computing device 140. Thus, a medical professional may view and interpret the ECG from wearable device 110 at 320 in order to determine whether the ECG has a normal or abnormal pattern. Moreover, the ECG taken by wearable device 110 may be visually analyzed at 320. Such personal analysis at 320 may advantageously allow method 300 to be agnostic with respect to the type of wearable device used to take the ECG at 310. Method 300 may take ECGs with various types of wearable devices at 310, and the ECGs may be reviewed at 320 without regard to the particular type of wearable device used to take the ECG at 310.

At 330, it may be determined whether the interpretation of the ECG from 320 indicates an irregularity, such as one or more of: (1) a cardiac rhythm disturbance, such as atrial fibrillation or ventricular tachycardia; (2) inadequate coronary artery blood flow, such as myocardial ischemia or myocardial infarction; and (3) electrolyte disturbances, as hypokalemia and hyperkalemia. At 340, the interpretation of the ECG from 320 may be reported to the user of wearable device 110 when the interpretation of the ECG does not indicate an irregularity at 340. Thus, data corresponding to the interpretation of the ECG from 320 may be transmitted from interpretation provider computing device 140 to various components of system 100, such as wearable device 110, personal computing device 120, and/or server 130, via network 150. The data corresponding to the interpretation of the ECG from 320 may be received at wearable device 110 and/or personal computing device 120 such that the user of wearable device 110 may review the interpretation of the ECG from 320. As a particular example, the interpretation of the ECG may be reported to the user of wearable device 110 as a pdf or other file format sent to the wearable device 110 and/or personal computing device 120 for viewing by the user of wearable device 110.

At 350, the interpretation of the ECG from 320 may also be reported to the user of wearable device 110 when the interpretation of the ECG indicates the irregularity at 340. Moreover, a referral to a medical service provider may also be transmitted to the user of wearable device 110 when the interpretation of the ECG indicates the irregularity at 340. Thus, data corresponding to the interpretation of the ECG from 320 as well as a referral to a medical service provider may be transmitted to wearable device 110 and/or personal computing device 120 such that the user of wearable device 110 may review the interpretation of the ECG from 320 and contact the medical service provider for follow-up treatment and/or testing. The referral may include a list of cardiologists and/or electrophysiologists located proximate the user. Thus, the user of wearable device 110 may be notified of nearby cardiologists and/or electrophysiologists capable of follow-up treatment and/or testing to address the irregularity detected in the ECG. Method 300 may also include transmitting one or both of the ECG from 310 and the interpretation of the ECG from 320 to the medical service provider in order to assist the medical service provider with follow-up treatment and/or testing to in response to the irregularity detected in the ECG.

As may be seen from the above, method 300 may advantageously provide the user of wearable device 110 with a referral to a medical service provider located nearby the user of wearable device 110 when an irregularity is found in the ECG taken by the wearable device 110. Method 300 may advantageously be agnostic with respect to the type of wearable device taking the ECG. For example, method 300 may allow for interpretation of ECGs from Apple Watches, Fitbits, Samsung Galaxy Watches, Withings ScanWatches, etc. Thus, e.g., ECGs from a variety of wearable devices may be interpreted to detect irregularities and make referrals to medical service providers.

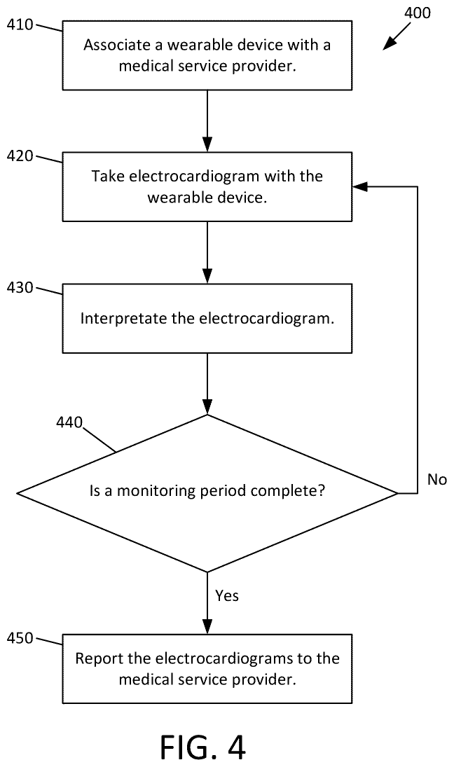

FIG. 4 is a flow diagram of a method 400 for heart monitoring according to an example embodiment of the present subject matter. Method 400 will generally be described with reference to system 100 described with reference to FIGS. 1 and 2. For instance, method 400 may be at least partially executed by system 100, such as by one or more of wearable device 110, personal computing device 120, server 130, and interpretation provider computing device 140. However, method 400 may be suitable for use with any other suitable type of system. In addition, although FIG. 4 depict steps performed in a particular order for purposes of illustration and discussion, the methods and algorithms discussed herein are not limited to any particular order or arrangement. One skilled in the art, using the disclosures provided herein, will appreciate that various steps of the methods and algorithms disclosed herein can be omitted, rearranged, combined, and/or adapted in various ways without deviating from the scope of the present disclosure.

At 410, wearable device 110 may be associated with a medical service provider, such as a cariologist, an electrophysiologist, an internist, a general practitioner, a cardiology practice group, hospital group, etc. For example, the user of wearable device 110 may be a patient of the medical service provider, and system 100 may be configured to send data, such as ECGs from wearable device 110 and/or interpretations of the ECGs, to the medical service provider after wearable device 110 is associated with the medical service provider. The association of wearable device 110 with the medical service provider may include a consent from the user of wearable device 110 to send medical information collected by system 100 to the medical service provider. In addition, at 410, user information for wearable device 110 may be sent to the medical service provider. For example, the user information for wearable device 110 may include one or more of a name of the user of wearable device 110, a location of the user of wearable device 110, an age of the user, a sex of the user, an address of the user, a zip code of the user, a medical history of the user, etc. Such user information may assist with interpretation of electrocardiograms (ECGs) and/or providing medical treatment for the user of wearable device 110.

At 420, an ECG may be taken with wearable device 110. For example, electrical heart sensor 117 may measure electrical activity of the heart of the user of wearable device 110, e.g., via a single lead of electrical heart sensor 117 contacting the skin of the user. The ECG may be taken at 410 in response to a user input at wearable device 110. Thus, the user of wearable device 110 may select when the ECG is taken at 410. In alternative example embodiments, the ECG may be automatically taken at 410, e.g., according to a predetermined schedule, in response to a sensed parameter, etc. Data corresponding to the ECG taken by wearable device 110 at 410 may be transmitted from wearable device 110 to various components of system 100, such as personal computing device 120, server 130, and/or an interpretation provider computing device 140, via network 150. The data corresponding to the ECG taken by wearable device 110 at 410 may be received at interpretation provider computing device 140.

At 430, the ECG taken by wearable device 110 at 420 may be interpreted. For example, a cardiologist, an electrophysiologist, a cardiac nurse practitioner, a cardiac physician assistant, or other interpretation provider may view and interpret the ECG from wearable device 110 at interpretation provider computing device 140. Thus, a trained medical professional may view and interpret the ECG from wearable device 110 at 430 in order to determine whether the ECG has a normal or abnormal pattern. Moreover, the ECG taken by wearable device 110 may be visually analyzed at 430. Such personal analysis at 430 may advantageously allow method 400 to be agnostic with respect to the type of wearable device used to take the ECG at 420. Method 400 may thus take ECGs with various types of wearable devices at 420, and the ECGs may be reviewed at 430 without regard to the particular type of wearable device used to take the ECG at 420.

The medical service provider associated with the wearable device 110 may be alerted when the interpretation of the ECG from 430 indicates an irregularity, such as one or more of: (1) a cardiac rhythm disturbance, such as atrial fibrillation or ventricular tachycardia; (2) inadequate coronary artery blood flow, such as myocardial ischemia or myocardial infarction; and (3) electrolyte disturbances, as hypokalemia and hyperkalemia. Moreover, data corresponding to the ECG taken at 420 and/or the interpretation of the ECG from 430 may be transmitted to the medical service provider, e.g., so that the medical service provider can schedule follow-up treatment and/or testing. Data corresponding to the ECG taken at 420 and/or the interpretation of the ECG from 430 may also be transmitted to the user of wearable device 110 (e.g., to wearable device 110 and/or personal computing device 120) such that the user of wearable device 110 may review the interpretation of the ECG from 320 and/or contact the medical service provider for follow-up treatment and/or testing. As a particular example, the interpretation of the ECG may be reported to the medical service provider and/or the user of wearable device 110 as a pdf or other file format.

The interpretation of the ECG from 430 may also be reported to the medical service provider and/or the user of wearable device 110 when the interpretation of the ECG does not indicate an irregularity at 430. Thus, data corresponding to the interpretation of the ECG from 430 may be transmitted from interpretation provider computing device 140 to various components of system 100, such as wearable device 110, personal computing device 120, and/or server 130, via network 150. Data corresponding to the ECG taken at 420 and/or the interpretation of the ECG from 430 may be transmitted to the medical service provider, e.g., so that the medical service provider can review the ECG and/or interpretation. Data corresponding to the ECG taken at 420 and/or the interpretation of the ECG from 430 may also be transmitted to the user of wearable device 110 (e.g., to wearable device 110 and/or personal computing device 120).

At 440, it is determined whether a monitoring period is complete. For example, system 100 may be configured to collect ECGs from wearable device 110 for a period of time. The period of time may be selected to conform to a billable period. For example, the period of time may be no less than sixteen (16) days in certain example embodiments. Thus, when the monitoring period is not complete at 440, method 400 may loop back to 420 and continue to take ECGs with wearable device 110 and such ECGs may be interpreted by the interpretation provider at 430. Conversely, when the monitoring period is complete, method 400 may continue to 450, e.g., because ECGs have been taken with wearable device 110 and interpreted for the billable period.

By taking ECGs at 420 and interpreting the ECGs at 430 for the period of time, method 400 may include taking a plurality of ECGs with wearable device 110 and interpreting the plurality of ECGs. Moreover, each of the plurality of ECGs may be interpreted by the interpretation provider during the period of time. In certain example embodiments, an ECG may be taken at least daily during the monitoring period. Thus, e.g., no less than sixteen ECGs may be taken with wearable device 110 during method 400. However, it will be understood that other suitable numbers of ECGs may be taken during the monitoring period. For instance, an ECG may be taken with wearable device 110 every twelve hours, every six hours, every hour, etc. during the monitoring period.

At 450, a billable report for the ECGs taken with wearable device and the interpretations of the ECGs may be transmitted to the medical service provider associated with the wearable device 110, e.g., after the period of time is complete. The billable report may include a summary of the interpretations of the ECGs. Thus, the ECGs taken as part of method 400 during the period of time may be listed, compilated, summarized, etc. for the medical service provider in the billable report. The billable report may also include a summary of face-to-face interactions between the user of wearable device 110 and the medical service provider. For example, the user of wearable device 110 and the medical service provider may meet in person for no less than twenty (20) minutes during the period of time, and the face-to-face interactions between the user of wearable device 110 and the medical service provider period of time may be listed, compilated, summarized, etc. within the billable report. The billable report may be submitted to an insurance provider for the user of wearable device 110. Thus, the medical service provider may advantageously be compensated for the heart health monitoring provided by the medical service provider during method 400.

As may be seen from the above, method 400 may advantageously provide the medical service provider with a billable report of ECGs taken with the wearable device 110. Method 400 may advantageously be agnostic with respect to the type of wearable device taking the ECGs. For example, method 400 may allow for interpretation of ECGs from Apple Watches, Fitbits, Samsung Galaxy Watches, Withings Scan Watches, etc. Thus, e.g., the medical service provider may advantageously allow patients to take ECGs from a variety of wearable devices to potentially detect irregularities and monitor heart health. Moreover, the medical service provider may not be limited to monitoring heart health of patients with a particular type of wearable device. In addition, the medical service provider may submit the billable report to an insurance company of the patient in order to be compensated for monitoring the hearth health of the user of wearable device 110, without regard to the particular type of wearable device owned or used by the patient. Accordingly, method 400 may be used by the medical service provider to monitor heart health for a plurality of patients, each taking ECGs with one of a variety of wearable devices.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they include structural elements that do not differ from the literal language of the claims, or if they include equivalent structural elements with insubstantial differences from the literal languages of the claims.

What is claimed is:

1. A method for interpreting electrocardiograms, comprising:
associating a wearable device with a medical service provider;
receiving data corresponding to a plurality of electrocardiograms over a period of time, each of the plurality of electrocardiograms taken by the wearable device, the period of time being no less than sixteen days;
receiving data corresponding to an interpretation of each of the plurality of electrocardiograms by an interpretation provider during the period of time; and
transmitting data corresponding to a billable report for the interpretation of each of the plurality of electrocardiograms to the medical service provider after the period of time.

2. The method of claim 1, further comprising transmitting the data corresponding to the plurality of electrocardiograms to the medical service provider during the period of time.

3. The method of claim 1, further comprising transmitting the data corresponding to the plurality of electrocardiograms to the interpretation provider during the period of time.

4. The method of claim 1, further comprising transmitting the data corresponding to the interpretation of each of the plurality of electrocardiograms by the interpretation provider to the medical service provider during the period of time.

5. The method of claim 1, wherein each of the plurality of electrocardiograms is taken in response to a user input at the wearable device.

6. The method of claim 1, wherein each of the plurality of electrocardiograms is automatically taken according to a predefined schedule.

7. The method of claim 1, wherein the plurality of electrocardiograms comprises no less than sixteen electrocardiograms.

8. The method of claim 1, wherein the billable report comprises a summary of the interpretation of each of the plurality of electrocardiograms.

9. The method of claim 1, wherein the billable report comprises a summary of face-to-face interactions between a user of the wearable device and the medical service provider.

10. A computing system, comprising:
one or more processors; and
one or more non-transitory computer-readable media that store instructions that, when executed by the one or more processors, cause the computing system to perform operations, the operations comprising
associating a wearable device with a medical service provider,
receiving data corresponding to a plurality of electrocardiograms over a period of time, each of the plurality of electrocardiograms taken by the wearable device, the period of time being no less than sixteen days,
receiving data corresponding to an interpretation of each of the plurality of electrocardiograms by an interpretation provider during the period of time, and
transmitting data corresponding to a billable report for the interpretation of each of the plurality of electrocardiograms to the medical service provider after the period of time.

11. The computing system of claim 10, further comprising transmitting the data corresponding to the plurality of electrocardiograms to the medical service provider during the period of time.

12. The computing system of claim 10, further comprising transmitting the data corresponding to the plurality of electrocardiograms to the interpretation provider during the period of time.

13. The computing system of claim 10, further comprising transmitting the data corresponding to the interpretation of each of the plurality of electrocardiograms by the interpretation provider to the medical service provider during the period of time.

14. The computing system of claim 10, wherein each of the plurality of electrocardiograms is taken in response to a user input at the wearable device.

15. The computing system of claim 10, wherein each of the plurality of electrocardiograms is automatically taken according to a predefined schedule.

16. The computing system of claim 10, wherein the plurality of electrocardiograms comprises no less than sixteen electrocardiograms.

17. The computing system of claim 10, wherein the billable report comprises a summary of the interpretation of each of the plurality of electrocardiograms.

18. The computing system of claim 10, wherein the billable report comprises a summary of face-to-face interactions between a user of the wearable device and the medical service provider.

19. A method for interpreting electrocardiograms, comprising:
receiving data corresponding to an electrocardiogram taken by a wearable device;
transmitting data corresponding to an interpretation of the electrocardiogram by an interpretation provider; and
transmitting data corresponding to a referral to a medical service provider in response to the interpretation of the electrocardiogram indicating an irregularity.

20. The method of claim 19, further comprising receiving data corresponding to user information prior to receiving the electrocardiogram,
wherein the user information comprises one or more of a location of the user, an age of the user, a sex of the user, an address of the user, a zip code of the user, and a medical history of the user, and
wherein the referral comprises a list of a plurality of cardiologists and/or electrophysiologists located proximate the user.
